(19) United States
(12) Patent Application Publication
(10) Pub. No.: US 2024/0325023 A1
(43) Pub. Date: Oct. 3, 2024

(54) HEMORROHOIDECTOMY LINEAR STAPLER" to "LINEAR STAPLE FOR FOR END - END ANASTOMOSIS PROCEDURES".

(71) Applicant: KING FAISAL UNIVERSITY, Al-Ahsa (SA)

(72) Inventor: Abdulrahman SALEH ALMULHIM, AL-AHSA (SA)

(21) Appl. No.: 18/221,119

(22) Filed: Jul. 12, 2023

Related U.S. Application Data
(62) Division of application No. 18/127,255, filed on Mar. 28, 2023.

(51) Int. Cl. A61B 17/10; A61B 17/32

(52) U.S. Cl. CPC A61B 17/105; A61B 17/320016; A61B 2017/00818; A61B 17/068; A61B 2017/3452

(57) ABSTRACT

A system and method for performing a hemorrhoidectomy using a laparoscope, endoscope, colonoscope, or gastroscope for the deployment of a surgical fastener into the area of the removed hemorrhoid. Once optimally positioned, the surgical fastener is applied using an applicator and the adjacent tissues are sealed.

### BACKGROUND

### 1. Field

The present disclosure relates to a surgical fastener for use with a variety of surgical fastener applying devices suitable for performing a variety of surgical procedures, including but not limited to curved or circular anastomosis and/or treatment to internal walls of hollow tissue organs, and treating internal hemorrhoids in the rectum area.

### 2. Description of the Related Art

Hemorrhoidal disease is a common disorder requiring surgical intervention in approximately 10% of cases. The overall prevalence is unknown because asymptomatic patients are less likely to seek medical help. Some sources estimate the incidence of symptomatic patients in the U.S is 4.4%, with patients between the ages of 45 to 65 years old being most affected. Surgical hemorrhoidectomy is necessary for large third- and fourth-degree hemorrhoids when medical management has failed, when there is a large bulging external component, or when incarcerated internal hemorrhoids need immediate medical treatment.

Hemorrhoids are columns of vascular connective tissue within the anal submucosa aiding in maintaining continence and bulk to the anal canal. The pathophysiology of hemorrhoids is mostly unknown, but one theory states that hemorrhoids develop due to varicose veins in the anal canal. However, most healthcare providers reject this idea. Instead, the current thought is that hemorrhoids develop due to decay or deterioration of the vascular cushions. The three primary hemorrhoidal columns are in the left lateral, right anterolateral, and right posterolateral positions of the anal canal and can be either internal or external based on their location relative to the dentate line. Internal hemorrhoids can be further divided on a scale from I to IV based on the degree of prolapse, which also helps guide the treatment options. Patients presenting with symptomatic internal hemorrhoids complain of painless, bright red bleeding, described as streaks of blood in the stool, anal itching, pain, worrisome grape-like tissue prolapse, or a combination of these symptoms. External hemorrhoids are asymptomatic in most patients except for thrombosed external hemorrhoids, which cause significant pain due to their innervation by somatic nerves.

Hemorrhoids can have treatment with both medical and surgical interventions depending on their degree of prolapse and whether they are internal or external. The most effective treatment for recurrent, symptomatic grade III or IV hemorrhoids, is surgical excision. Surgical procedures primarily include closed, also called Ferguson, hemorrhoidectomy, which is the most common technique in the United States, or the open, also called Milligan-Morgan, hemorrhoidectomy, used in the United Kingdom and Europe. Accordingly, when resorting to a surgical solution, rectal hemorrhoid treatment often employs a circular fastening device consisting of an anvil head and a fastener holding component, where the head and the holding component are inserted into the rectum. Thereafter, a purse-string suture is used to pull the internal hemorrhoidal tissue forward such that the anvil head and the fastener holding component can clamp the hemorrhoidal tissue therebetween. The fastening device is then fired to remove the hemorrhoidal tissue and fasten the cut tissue. However, a significant drawback with this procedure is that there is only so much hemorrhoidal tissue that can be drawn into and around the anvil head for removal.

What is needed is a surgical stapler and/or apparatus that is less complex and can more quickly perform a surgical procedure than existing staplers. And, in addition, there exists a need to attempt to reduce the trauma caused to the patient during the surgical procedures described above.

### SUMMARY

The laparoscopic method and device for performing a hemorrhoidectomy is provided to treat hemorrhoids in a more minimally invasive and efficient way. In this regard, the present subject matter relates to a device that fits on a standard laparoscope such that it can be deployed near the affected area of the anus and/or rectum in order to execute the steps of the procedure. It includes a cutting element such as a knife for cutting away adjacent tissue and a fastening apparatus which attaches a fastener or a series of fasteners to the treated area. In practice, firing of the fastening device by compressing the trigger on the handle will result in removal of the hemorrhoidal tissue and fastening the cut tissue.

In an embodiment, the present subject matter relates to a laparoscopic method for performing a hemorrhoidectomy in a patient, comprising: positioning a distal end of a laparoscope near an affected area in a surgical field of the patient where said affected area includes a hemorrhoid wherein said hemorrhoid is to be surgically removed; positioning an operative tool of a surgical fastener applicator near said hemorrhoid in said affected area wherein said operative tool includes a knife and a surgical fastener cartridge; actuating the knife to cut away tissue of said hemorrhoid from said affected area along a cut line; removing said cut away tissue of said hemorrhoid from said affected area of said surgical field; and firing a single surgical fastener from said surgical fastener cartridge of said operative tool, said single surgical fastener fastening together tissues adjacent to said cut away tissue of said hemorrhoid.

In another embodiment, the present subject matter relates to a device for use with a laparoscope for performing a hemorrhoidectomy, comprising: a handle for positioning a distal end of a laparoscope near an affected area in a surgical field of a patient where said affected area includes a hemorrhoid wherein said hemorrhoid is to be surgically removed; an elongated shaft for positioning an operative tool of a surgical fastener applicator near said hemorrhoid in said affected area wherein said operative tool includes a knife and a surgical fastener cartridge wherein said operative tool actuates the knife to cut away tissue of said hemorrhoid from said affected area along a cut line and remove said cut away tissue of said hemorrhoid from said affected area of said surgical field, and the surgical fastener cartridge disposed in said operative tool fires off a single surgical fastener, said single surgical fastener fastening together tissues adjacent to said cut away tissue of said hemorrhoid in use.

These and other features of the present subject matter will become readily apparent upon further review of the following specification.

### BRIEF DESCRIPTION OF THE DRAWINGS

FIG. 1 is a perspective view of a laparoscopic instrument with a linear stapler and cutting tool.

Similar reference characters denote corresponding features consistently throughout the attached drawings.

### DETAILED DESCRIPTION OF EMBODIMENTS

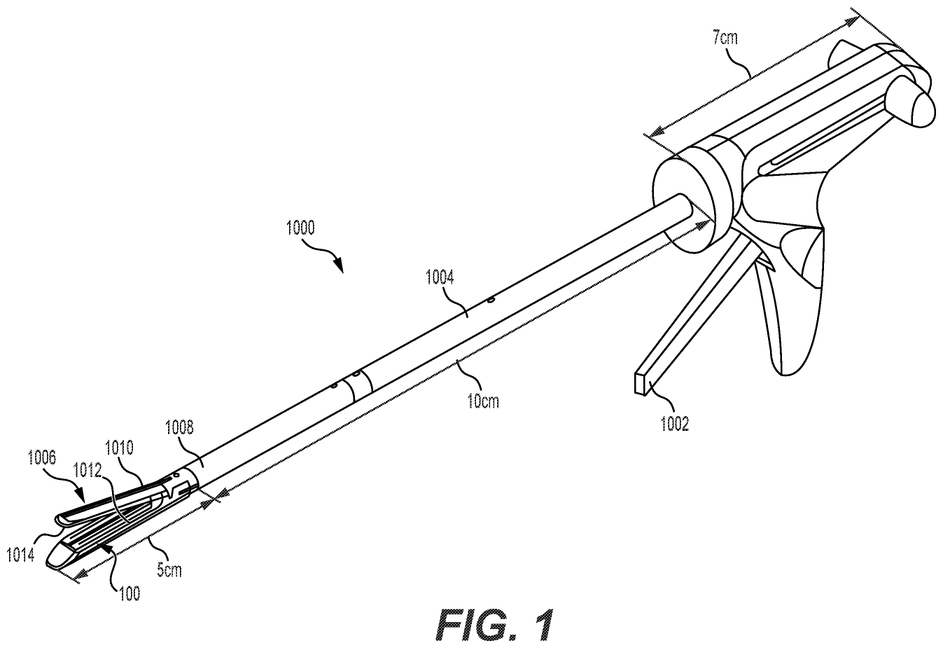

An exemplary embodiment of the presently disclosed hemorrhoidectomy linear stapler will now be described in detail with reference to FIG. 1. According to the present disclosure, it is envisioned that the surgical fasteners or staples disclosed herein may be utilized with various surgical fastening or stapling instruments, such as circular fasteners or staplers, linear fasteners or staplers, and transverse fasteners or staplers, including open and laparoscopic/endoscopic fasteners or staplers.

According to the present description, the term “proximal” will refer to the end of the device including the fastener or staple cartridge assembly, fastening or stapling device, or component thereof while the term distal will refer to the end that is farthest from the operator, as is traditional and conventional in the art.

In addition, the term “surgical fastener” should be understood to include any substantially rigid structure formed of a biocompatible material that is suitable for the intended purpose of joining together adjacent tissue portions including but not being limited to surgical staples, clips, two-part fasteners and the like.

With reference to FIG. 1, a surgical fastener applying apparatus (1000) that employs a surgical fastener cartridge (100) holding, e.g., a plurality of staples is illustrated. In this regard, the surgical fastener cartridge (100) can have a length of about 1.5 cm. The surgical fastener applying apparatus (1000) is used to sequentially apply a plurality of surgical fasteners to a patient's tissue. The surgical fastener apparatus (1000) may be configured for use, subsequent sterilization, and reuse, or may be configured for a single use. Surgical fastener apparatus (1000) includes a handle (1002) which is about 7 cm in length, an elongated shaft or laparoscopic portion (1004) extending distally therefrom for about 10 cm, and an operative tool (1006) coupled to the distal end (1008) of the elongated shaft (1004) having a length of about 5 cm. In general the operative tool (1006) is adapted to clamp, sequentially fasten or staple together, and sever adjacent tissue segments along a cut-line.

Operative tool (1006) includes a pair of opposed jaws (1010), (1012) pivotally coupled to one another and respectively including an anvil member (1014) that is approximated relative to the cartridge (100) during use. In this regard, the operative tool (1006) can have a knife or other cutting element as a first jaw (1010) of the pair of opposed jaws said surgical fastener cartridge (100) disposed within a second jaw (1012) of the pair of opposed jaws.

The anvil member (1014) includes depressions that are aligned with, and/or are in registration with slots defined in the surgical fastener cartridge (100), through which the fasteners will emerge, in use, to effectuate formation. The fasteners or staples may be formed of a stainless steel wire of circular cross-section, although other materials and wire shapes are contemplated. For example, the fasteners or staplers may be formed entirely or partially of silver, titanium, and/or any other conductive or non-conductive material. While the fastener or staple is in one embodiment shown to have a circular cross-section throughout its length, it is envisioned that the fastener or stapler may have a variety of different cross sections including rectangular, oval, square, triangular, trapezoidal, etc. In general, each fastener or staple will have an open configuration of about 2.5 mm and a closed or collapsed configuration of about 1 mm, where the closed configuration is the configuration used for fastening or stapling function.

While surgical fastening apparatus (1000) is depicted for use as an apparatus for use in laparoscopic procedures for performing surgical anastomotic fastening or stapling of tissue, those skilled in the art will appreciate that cartridge (100), and/or the fasteners or staples contained therein, may be adapted for use with any surgical instrument capable for the intended purpose described herein. For example, the cartridge (100) may be adapted for use with an end-to-end anastomosis device (not shown) wherein the surgical fasteners or staples are arranged in substantially annular rows; and or a surgical stapling instrument (not shown) for use during a gastro-intestinal anastomotic stapling procedure wherein the surgical fasteners or staples are arranged in substantially linear rows.

In use, the present methods can include steps of positioning an operative tool of a surgical fastener applicator near a hemorrhoid in said an area of a patient wherein the operative tool includes a knife and a surgical fastener cartridge;
actuating the knife to cut away tissue of said hemorrhoid from said affected area along a cut line;
removing said cut away tissue of said hemorrhoid from said affected area of said surgical field; and
firing a single surgical fastener from said surgical fastener cartridge of said operative tool, said single surgical fastener fastening together tissues adjacent to said cut away tissue of said hemorrhoid.

In this regard, actuating the knife and/or firing the single surgical fastener will be effected by squeezing a trigger on the handle of the operative tool.

It is to be understood that the laparoscopic method and device for performing a hemorrhoidectomy is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1-8. (canceled)

9. An end-to-end anastomosis device for performing a surgical anastomotic fastening, comprising:
a handle for positioning a distal end of a surgical fastener applicator near an affected area in a surgical field of a patient where said affected area includes tubular tissue segments undergoing an anastomotic fastening;
an elongated shaft for positioning an operative tool of the surgical fastener applicator near said tubular tissue segments in said affected area wherein said operative tool includes a knife and a surgical fastener cartridge, wherein said operative tool has a pair of opposed jaws which are pivotably coupled to one another and respectively include an anvil member, and wherein said operative tool actuates the knife to cut away tissue from said tubular tissue segments included in said affected area along a cut line and remove said cut away tissue from said tubular tissue segments included in said affected area of said surgical field; and
the surgical fastener cartridge disposed in said operative tool fires off a single surgical fastener while the pair of opposing jaws grasp said tubular tissue segments together, said single surgical fastener fastening together said tubular tissue segments adjacent to said cut away tissue in use.

10. (canceled)

11. The end-to-end anastomosis device for performing a surgical anastomotic fastening as recited in claim 9, wherein said operative tool is about 5 cm in length and has the knife as a first jaw of the pair of opposed jaws.

12. The end-to-end anastomosis device for performing a surgical anastomotic fastening as recited in claim 11, wherein said operative tool has said surgical fastener cartridge disposed within a second jaw of the pair of opposed jaws.

13. The end-to-end anastomosis device for performing a surgical anastomotic fastening as recited in claim 12, wherein said single surgical fastener is a staple wherein said staple has an open configuration of about 2.5 mm in height and a closed or collapsed configuration of about 1 mm in height.

14. The end-to-end anastomosis device for performing a surgical anastomotic fastening as recited in claim 9, wherein the anvil member includes depressions that are aligned with, and/or are in registration with, slots defined in the surgical fastener cartridge, through which said single surgical fastener will emerge, to effectuate formation.
